(19) United States
(12) Patent Application Publication
(10) Pub. No.: US 2020/0163585 A1
(43) Pub. Date: May 28, 2020

(54) METHOD FOR THE EARLY IDENTIFICATION OF RECURRENCES OF CHRONIC OBSTRUCTIVE PULMONARY DISEASE

(71) Applicants: RESTECH S.R.L., Milan (IT); POLITECNICO DI MILANO, Milan (IT)

(72) Inventors: Alessandro GOBBI, Bergamo (IT); Pasquale Pio POMPILIO, Milan (IT); Raffaele DELLACÀ, Valmorea (IT)

(21) Appl. No.: 16/637,991

(22) PCT Filed: Aug. 3, 2018
(86) PCT No.: PCT/IB2018/055857

§ 371 Date: Feb. 10, 2020

(30) Foreign Application Priority Data
Aug. 11, 2017 (IT) 102017000093172

(51) Int. Cl. A61B 5/085; G06F 17/18; G16H 50/30; A61B 5/08; A61B 5/00

(52) U.S. Cl. CPC A61B 5/085; G06F 17/18; A61B 5/7275; A61B 5/0803; A61B 5/0816; G16H 50/30

(57) ABSTRACT

A method for the early identification of recurrences of chronic obstructive pulmonary disease comprising the following steps of; measuring, with a predefined time frequency, a plurality of parameters that define the pulmonary function of a patient by means of the forced oscillation technique (FOT); calculating the trend of said plurality of parameters in a predefined time period; identifying an impending recurrence by comparing the parameters describing said trend of said plurality of parameters with predefined thresholds; where the step of calculating the trend of said plurality of parameters is achieved by calculation of an N order polynomial regression model; and the step of identifying an impending recurrence by comparing said parameters describing said trend with predefined thresholds comprises the step of comparing at least one coefficient of the N order polynomial regression with predefined thresholds.

### DISCLOSURE

The present invention refers to a method for the early identification of recurrences in patients suffering from chronic obstructive pulmonary disease.

Chronic obstructive pulmonary disease (COPD) is a chronic respiratory disease characterized by persistent symptoms such as dyspnea, chronic coughing and expectoration and by persistent airflow limitation (GOLD 2017). Common risk factors include prolonged exposure to noxious particles and/or gases, such as cigarette smoke. The progression of COPD is characterized by stable periods interrupted by recurrences, namely acute deteriorations of the symptoms and the underlying inflammatory process which, in the most serious cases, can require hospitalization of the patient (Vogelmeier et al., 2017).

The frequency of the recurrence episodes has important consequences for the clinical history of the patient, accelerating functional decline of the lungs, increasing the risk of death, reducing the quality of life and increasing the social and economic costs associated with the pathology.

The evidence that early therapeutic intervention on the recurrence episodes can help to reduce their impact on the patients' health (Wilkinson, Donaldson, Hurst, Seemungal, & Wedzicha, 2004), together with the necessity to optimize the management of patients suffering from COPD, has stimulated the development of care models based on home monitoring programs. The majority of the programs proposed are based on the use of daily questionnaires for recording worsening of the symptoms perceived by the patients in combination with medical teleconsulting systems and patient education. Although these programs have demonstrated effectiveness in reducing hospitalizations and the number of patients accessing A&E due to recurrences of COPD (McLean et al., 2012), they have not been applied on a large scale due to the high implementation costs required.

An alternative approach consists in the combination of measurements of physiological parameters that can be performed by the COPD patient at home, without direct medical supervision, with automatic algorithms that are able to identify the recurrences early starting from analysis of the measurements performed. The medical personnel are therefore alerted only if the algorithm has identified a suspected deterioration in the state of health of one of the COPD patients being treated who, consequently, can be immediately contacted to verify his/her state of health and/or to optimize the course of treatment.

Since said approach does not require continuous review of the measurements taken by the medical personnel, it would allow the management of a large number of patients by a restricted medical team, thus guaranteeing implementation on a large scale.

The experimental studies in which said approach has been studied used measurements of cardiac frequency and blood oxygen saturation (measured by means of portable pulsometers), alone or in combination with mechanical respiratory measurements (with portable spirometers). Said studies have not demonstrated adequate effectiveness in improving the management of patients suffering from COPD during a recurrence (Ringbaek et al., 2015; Vianello et al., 2016).

The object of the present invention is to provide a method for early identification of recurrences of COPD, using respiratory function parameters measured by means of the forced oscillation technique (FOT).

In accordance with the present invention, said object and others are achieved by a method for the early identification of recurrences of chronic obstructive pulmonary disease comprising the following steps: measuring, with a predetermined time frequency, a plurality of parameters that define the pulmonary function of a patient by means of the forced oscillation technique (FOT); calculating the trend of said plurality of parameters in a predefined time period; identifying an impending recurrence by comparing the parameters describing said trend of said plurality of parameters with predefined thresholds; where the step of calculating the trend of said plurality of parameters is achieved by calculating an N order polynomial regression model; and the step of identifying an impending recurrence by comparing said parameters describing said trend with predefined thresholds comprises the step of comparing at least one coefficient of the N order polynomial regression with predefined thresholds.

Further characteristics of the invention are described in the dependent claims.

The forced oscillation technique (FOT) is a non-invasive method for measuring the mechanical properties of the airways and lungs based on the recording of pressure and flow to the patient's mouth during the application of a low-pressure external stimulus oscillating at a frequency higher than that of spontaneous breathing. (Dubois, Brody, Lewis and Burgess, 1956). This characteristic allows the measurement to be performed during spontaneous breathing, therefore making it ideal for remote monitoring applications, without supervision, of the respiratory parameters as demonstrated for example in the pilot studies of Dellaca et al. (Raffaele L. Dellaca, Gobbi, Pastena, Pedotti and Celli, 2010) and Gulotta et al. (Gulotta et al., AJRCCM, 2012).

During the FOT measurement, small oscillations in pressure (approximately 1-3 cmH2O peak-peak) at a single or composite frequency (usually between 4 and 40 Hz) are sent to the patient's lungs through the opening of the airways (nose and/or mouth) by using a mouthpiece or alternative interfaces such as nasal or facial masks. The response of the respiratory system is evaluated in terms of impedance (Zrs), which is the overall ratio between the pressure at the mouth and the airflow at the oscillation frequencies. The impedance Zrs is usually divided into its real component, the resistance (Rrs), and the imaginary component, the reactance (Xrs).

Rrs and Xrs can be analysed both in the time domain, i.e. during the respiration cycle (intra-breath analysis) and in the frequency domain (frequency analysis).

In the first case (intra-breath analysis) Rrs and Xrs are calculated at each breath, as described for example in Dellaca et al. (Dellaca et al., ERJ, 2004). Rrs and Xrs can therefore be presented both for each breath or as a mean of all the breaths of a given measurement. The intra-breath analysis allows Rrs and Xrs to be used to automatically exclude some breaths from the measurement mean if they are affected by artefacts, such as swallowing, coughing, etc. An example of said algorithm is described in Gobbi et al. (Gobbi et al., IEEE Telemed, 2009). Furthermore, with respect to the frequency analysis, in the intra-breath analysis the number of frequencies contained in the pressure stimulus is usually lower; this allows improvement of the signal-noise ratio and further separation of the contribution of inspiration and expiration of both the Rrs (obtaining the inspiratory resistance, Rinsp, and expiratory resistance, Rexp, respectively) and the Xrs (obtaining the inspiratory reactance, Xinsp, and expiratory reactance, Xexp, respectively) at each stimulus frequency. The results of the inspiratory and expiratory parameters can be reported for both each breath and as a mean of the breaths without artefacts contained in the measurement itself. For example, the mean difference between Xinsp and Xexp at 5 Hz within an FOT test is indicated by the symbol AXrs and has been shown to be associated with expiratory flow reduction (R. L. Dellaca et al., 2004), a condition that occurs in patients affected by severe or very serious COPD. Since an FOT measurement is performed during quiet breathing, from said measurement it is also possible to derive various respiratory pattern parameters, for example the current volume (V_{T}), the mean inspiratory and expiratory flows and times, the respiratory frequency and minute ventilation.

The characteristics and advantages of the present invention will be evident from the following detailed disclosure of a practical embodiment thereof, illustrated by way of non-limiting example in the accompanying drawings, in which:

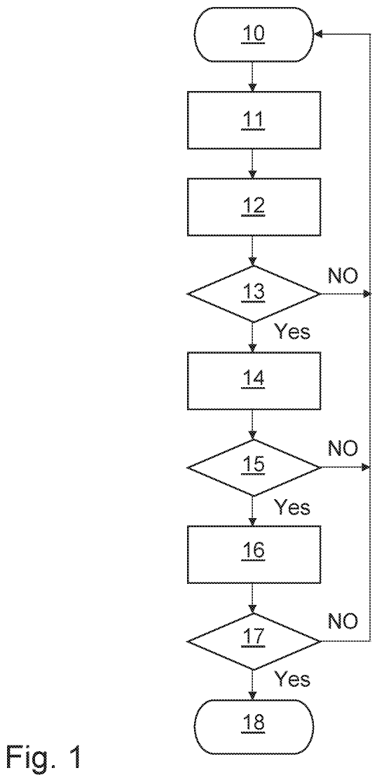

FIG. 1 shows a flow diagram of a method for early identification of recurrences of COPD, in accordance with the present invention;

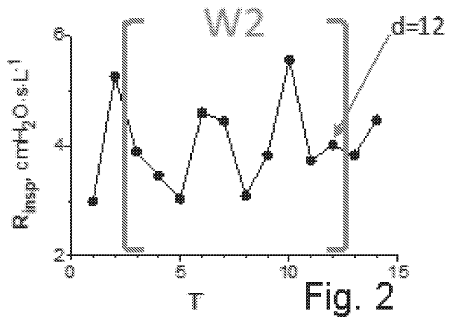

FIG. 2 shows a graph exemplifying Rinsp measurements taken on the various days indicated on the X axis and in the window W2.

Referring to the attached figure, a method for the early identification of recurrences of COPD, in accordance with the present invention, comprises the steps of initiating 10 the procedure; measuring 11, with a predefined time frequency, a certain number of parameters that define pulmonary function and the respiratory pattern of a patient by means of the FOT technique; for each new measurement available, collecting 12 the parameters measured, thus constituting the corresponding time series thereof; verifying 13 whether the adaptation period, calculated from the beginning of the time series, has finished, i.e. evaluating whether the number of measurements collected is higher than a first predefined number—if not, start again from the beginning 10, and if so, eliminate 14 the abnormal values; verifying 15 whether the number of measurements in a given time period (having eliminated the abnormal values) is higher than a predefined number—if not, start again from the beginning 10, and if so, calculate 16 the time trend of said parameters in a predetermined time period; verifying 17 whether the trend of the latter, evaluated by using appropriate statistical methods or mathematical models, is significantly higher or lower than predefined numbers—if not, start again from the beginning 10, and if so, an impending recurrence 18 has been predicted. Then start again from the beginning 10.

For the measurements 11 the patients are required to use an FOT device able to measure Rrs and Xrs separately during the inspiratory and expiratory phase, the derived parameters and the respiratory model parameters. Said device is composed of a Generator of stimuli at low pressure (<5 cmH2O), a set of pressure and flow sensors, a patient interface, a respiration circuit and a calculation unit that operates the pressure generator, collects the data from the sensors and uses them to calculate the pulmonary impedance, the derived parameters and the respiratory pattern parameters according to specific algorithms. An embodiment example of said device is described by Gobbi et al (Gobbi, Milesi, Govoni, Pedotti & Dellaca 2009).

During each measurement, the patients are required to wear a nose plug and adopt systems to reduce vibration of the cheeks (for example, by supporting them using their hands) while they breathe spontaneously through the device, for example for two minutes or until a predefined number of breaths has been recorded.

The parameters that define the pulmonary function of a patient measured by means of the FOT technique are one or more of the following: inspiratory resistance (Rinsp) measured at a frequency ranging between 2 and 10 Hz; inspiratory reactance (Xinsp) measured at a frequency ranging between 2 and 10 Hz; difference between inspiratory and expiratory reactance (AXrs) measured at a frequency between 2 and 10 Hz.

The respiratory pattern of a patient is described by the set of the following parameters: current volume (V_{T}), mean inspiratory (Ti) and expiratory times (Te), respiratory frequency (RR), respiratory duty cycle (Ti*RR), mean inspiratory (Vt/Ti) and expiratory flow (Vt/Te) and minute ventilation (Ve).

In one embodiment example of the method, the patient is required to perform one FOT measurement per day. The mean FOT and respiratory pattern parameters of each new daily measurement, calculated according to the intra-respiratory analysis method previously described, are collected 12 in the corresponding time series of the patient in question.

Since the measurement 11 requires the patient to breathe through the FOT device by means of a measurement interface, for example a mouthpiece, it is possible that the first measurements may not be usable due to adaptation of the patient to said interface. Said measurements should preferably be excluded. In one embodiment example of the method an adaptation period 13 of 8 days has been considered, so that the measurements contained in said time period are excluded from the following calculations. This passage is optional as it may not be necessary.

If an FOT measurement produces abnormal values, for example when carried out with an incorrect posture, without correct support of the cheeks, with a wrong positioning of the mouthpiece and/or of the nose plug, leaks around the measurement interface, due to obstruction of the filter by teeth or tongue, coughing, partial or total closure of the glottis, they must be eliminated 14 from the time series.

In one embodiment of the present invention, a method for detecting the abnormal values uses the normalized distance of one or more parameters calculated from the FOT measurement and the current daily respiratory pattern with corresponding mean value, calculated from the measurements available within a time window of predefined length which includes the current and past FOT measurements.

In particular it was considered that if the value V of a given parameter, calculated as shown in the following equation, is higher than a threshold value TR, the current FOT measurement OP must be considered abnormal and therefore discarded.

$$V = \frac{OP - m ( OP ( W 1 ) )}{m ( OP ( W 1 ) )} \geq TR \qquad ( 1 )$$

where:

m(OP(W1)) is considered the mean of the values of a given parameter measured within the window W1, and

W1 is a time window of predefined length containing the FOT measurements to be considered in the calculation, the new measurement and the past measurements.

Other approaches can be used to detect abnormal values in a time series of measurements and adapted for this application.

In a preferred embodiment of the present invention, the window W1 lasts 8 days and the threshold TR is equal to 0.5. The measurement is considered an abnormal value and will be ignored if the previous equation is verified for at least one of the following parameters: current volume V_{T}, inspiratory resistance Rinsp measured at 5 Hz, respiratory reactance Xinsp measured at 5 Hz.

It is preferably checked 15 that, after removal of the abnormal values, at least a predefined number of measurements are present in a given time period W2, in order to have a significant number of measurements. In a preferred embodiment of the present invention, the time window W2 was chosen equal to 10 days and the minimum number of FOT measurements that must be present in W2 equal to 5.

It is checked that in W2 there are at least X % measurements. For example, if X %=50% and W2=10 days, it must be checked that there are at least 5 measurements in W2.

The trends of all or a part of the FOT parameters and respiratory model are then calculated 16, by means of appropriate statistical methods or mathematical models and starting from the measurements available in the same time period W2. For example, a trend could be quantified, for each parameter in question, by means of an N order polynomial regression model relative to the measurements performed and previously processed considering: 1) the coefficients of the polynomial equation calculated (β₀ for the known term, β₁ for the coefficient of the first degree term, and so on), 2) the statistical significances (p-value) of each coefficient against the null hypothesis of being equal to zero, and 3) the correlation coefficient of the polynomial regression (r²).

For example, a linear regression model and the parameters Rinsp, Xinsp and DeltaXrs can be used, thus calculating β1_{Rinsp}, β1_{Xinsp} and β1_{deltaXrs}.

For each FOT parameter considered, it is evaluated whether the statistical regression model identifies a progression, calculating the probability of one or more parameters of the model β1 being different from zero, comparing said probability (also known as p-value), with a threshold, for example p<0.05. If this criterion is verified, it can be affirmed that the statistical model describes the progression of the parameter FOT sustained over time.

The overall goodness of the regression is then evaluated and its physiological significance. For measurement of the goodness of the regression, the correlation coefficient r2 can, for example, be used, which must be greater than a given threshold. The physiological significance of the regression is evaluated through a criterion applied to β1, which depends in turn on the FOT parameter considered. In this example, the criteria associated with the respective coefficients β1 are: β1_{Rinsp}>0, β1_{Xinsp}<0, β1_{deltaXrs}>0.

If the statistical regression model identifies a progression for a given FOT parameter and, simultaneously, the regression has a valid physiological significance and a high goodness level, the method assigns a value 1 to a corresponding trend parameter MI, which otherwise remains=0.

Therefore, for every parameter analysed, the trend is considered in the direction of worsening of the pathology if it is above or below a predefined threshold. If so, a value 1 is assigned to a corresponding trend parameter, MI_{P}. If not, the corresponding trend parameter MI_{P} is maintained at 0.

For example, we will therefore have three trend parameters MI_{Rinsp}, MI_{Xinsp} and MI_{deltaXrs} and each of them can assume the value 1 or remain at 0.

Lastly, a recurrence is scheduled by applying the following equation (2) which calculates a weighted sum of the trend parameters just processed:

$${\sum }_{p} {MI}_{p} * {w}_{p} \geq TH \qquad ( 2 )$$

where W_{P} (0≤W_{P}≤1) is a weight associated with the trend parameter MI_{P} of the parameter p in question and TH is a threshold.

In a preferred embodiment of the invention a linear regression model was applied (with N=1) to each of the following parameters: inspiratory resistance (Rinsp) measured at 5 Hz, absolute value of the inspiratory reactance (Xinsp) measured at 5 Hz, difference between inspiratory and expiratory reactance (ΔXrs) measured at 5 Hz.

Furthermore, for every parameter a value equal to 1 is assigned to the corresponding trend parameter MI_{P} if all the following conditions have been verified for the following values: the absolute value of the coefficient β₁ (slope of the regression line) must be greater than 0, the corresponding p-value must be less than 0.05 and the correlation coefficient of the polynomial regression (r²) must be greater than 0.4.

In one embodiment example of the present invention, the measurements performed on the patient are transferred to a microprocessor which carries out all the processing operations, according to the predefined program, and provides the final results to a viewer, identifying, in automatic mode, the presence of recurrences of chronic obstructive pulmonary disease.

An impending recurrence was identified using the weights W_{P} equal to 1 and the predefined threshold TH equal to 1, i.e. if the value calculated was greater than or equal to 1 as in the following equation:

1*MI_{Rinsp}+1*MI_{Xinsp}+1*MI_{Δxrs}≥1

The Applicant performed a test on 24 patients for 8 months taking daily measurements by means of FOT using a commercial instrument.

The characteristics of the 24 COPD patients monitored are shown in Table 1.

Throughout the study the patients were telephonically interviewed once a week to collect the following information: prescriptions and use of drugs and/or antibiotics, non-scheduled medical examinations and admissions to hospital.

The recurrences were classified as:

Slight: where there were changes in the current treatment or prescription of a short-acting bronchodilator,

Intermediate: where a corticosteroid was prescribed,

Severe: where systemic antibiotics were prescribed,

Very serious: when the patient was admitted to hospital.

In order to evaluate the performances of this method, all recurrences were grouped together, regardless of their severity. Furthermore, a sub-analysis was carried out only on severe and very serious recurrences, since the latter are considered the most critical events in terms of both the patient and the health service.

During the monitoring period, the patients reported a total of 26 recurrences, 13 of which were of slight or intermediate type, and 13 of severe or serious type. Of these, 18 (69%) were correctly identified by the method described above. Eight recurrences of slight or intermediate type (61.5%) and 10 recurrences of severe or very serious type (77%) were correctly identified by the method described above.

TABLE 1

| | |
|---|---|
| Sex (M/F) | 20/4 |
| Age (years) | 72.3 ± 6.9 |
| Height (cm) | 156.8 ± 7.0 |
| Weight (kg) | 74.9 ± 14.5 |
| Body mass index BMI (kg/m2) | 26.5 ± 4.3 |
| Maximum expiratory volume in 1 | 1.1 ± 0.3 |
| according to FEV1 (I) | |
| FEV1 (% pred) | 41.3 ± 12.4 |
| FEV1/FVC (% pred) | 42.1 ± 11.9 |

1. A system for the early identification of exacerbation of chronic obstructive pulmonary disease, comprising a microprocessor adapted to perform the following steps:
measuring, a plurality of measurements at predefined time frequency, a certain number of parameters that define the pulmonary function of a patient by means of the forced oscillation technique (FOT);
measuring simultaneously a certain number of parameters indicative the respiratory pattern, represented by at least one or more of the following: tidal volume (VT), mean inspiratory (Ti) and expiratory times (Te), respiratory frequency (RR), respiratory duty cycle (Ti*RR), mean inspiratory (Vt/Ti) and expiratory flow (Vt/Te) and a minute ventilation (Ve);
eliminating the measurements containing abnormal values of the above-mentioned parameters, by the step of comparing each measurement with a corresponding median value, calculated from the measurements available within a predetermined time window;
calculating the trend of said plurality of parameters in a predefined time period;
identifying an impending exacerbation by comparing the parameters describing said trend of said certain number of parameters with predefined thresholds;
where the step of calculating the trend of said certain number of parameters is achieved by calculation of an N order polynomial regression model; and the step of identifying an impending exacerbation by comparing said parameters describing said trends with predefined thresholds comprises the step of comparing at least one coefficient of the N order polynomial regression with predefined thresholds; wherein for each parameter of said certain number of parameters the deterioration trend of the pathology is assessed according to whether it is above or below a predefined threshold; and that it comprises the step of predicting a exacerbation by performing a weighted sum of said trend parameters (MIP).

2. The system according to claim 1 characterized in that the measurement of the pulmonary function of a patient by means of the forced oscillation technique (FOT) is performed at least once every two days.

3. The system according to claim 1, characterized in that the parameters that define the pulmonary function are represented by one or more of the following: inspiratory resistance (Rinsp) measured at a frequency ranging between 2 and 10 Hz; inspiratory reactance (Xinsp) measured at a frequency ranging between 2 and 10 Hz; difference between inspiratory and expiratory reactance (ΔXrs) measured at a frequency ranging between 2 and 10 Hz.

4. The system according to claim 1, characterized in that the step of eliminating the measurements containing abnormal values comprises the step of eliminating all the measurements taken in a given time period from the beginning of the measurements.

5. The system according to claim 1 characterized in that after the step of eliminating the measurements containing abnormal values, there is the step of verifying whether the number of remaining measurements is higher than a predefined number.

6. The system according to claim 1 characterized in that the step of eliminating the abnormal values of the above-mentioned parameters comprises the step of considering that if the value V of a given parameter, calculated as shown in the following equation, is higher than a threshold value TR, the current FOT measurement OP must be considered abnormal and therefore discarded as the following equation
$$V = \frac{OP - m ( OP ( W 1 ) )}{m ( OP ( W 1 ) )} \geq TR$$
where:
m (OP(W1)) is considered the median of the values of a given parameter measured within the window W1, and
W1 is a time window of predefined length containing the FOT measurements to be considered in the calculation.

7. The system according to claim 1 characterized in that the step of predicting a exacerbation by performing a weighted sum of said trend parameters is calculated as
$${\sum }_{p} MIp * Wp \geq TH$$
where W_{P} (0<W_{P}<1) is a weight associated with the trend parameter MI_{P} of the parameter p in question and TH is a threshold.

8. A method for the early identification of exacerbation of chronic obstructive pulmonary disease, wherein a microprocessor carries out all the processing operations, comprising the following steps of:
measuring a plurality of measurements, at predefined time frequency, a certain number of parameters that define the pulmonary function of a patient by means of the forced oscillation technique (FOT); measuring simultaneously a certain number of parameters indicative the respiratory pattern, represented by at least one or more of the following: tidal volume (VT), mean inspiratory (Ti) and expiratory times (Te), respiratory frequency (RR), respiratory duty cycle (Ti*RR), mean inspiratory (Vt/Ti) and expiratory flow (Vt/Te) and a minute ventilation (Ve);
eliminating the measurements containing abnormal values of the above-mentioned parameters, by the step of comparing each measurement with corresponding median value, calculated from the measurements available within a predetermined time window;
calculating the trends of said certain number of parameters in a predefined time period;
identifying an impending exacerbation by comparing the parameters describing said trend of said certain number of parameters with predefined thresholds;
where the step of calculating the trends of said certain number of parameters is achieved by calculation of an N order polynomial regression model; and the step of identifying an impending exacerbation by comparing said parameters describing said trends with predefined thresholds comprises the step of comparing at least one coefficient of the N order polynomial regression with predefined thresholds; wherein for each parameter of said certain number of parameters the deterioration trend of the pathology is assessed according to whether it is above or below a predefined threshold; and that it comprises the step of predicting a exacerbation by performing a weighted sum of said trend parameters (MI_{P}).

9. A computer program adapted to perform the method for the early identification of exacerbation of chronic obstructive pulmonary disease according to claim 8 when run on a computer.

10. A method for the early identification of exacerbation of chronic obstructive pulmonary disease comprising the following steps of:
measuring a plurality of measurements, at predefined time frequency, a certain number of parameters that define the pulmonary function of a patient by means of the forced oscillation technique (FOT);
measuring simultaneously a certain number of parameters indicative the respiratory pattern, represented by at least one or more of the following: tidal volume (V_{T}), mean inspiratory (Tᵢ) and expiratory times (Te), respiratory frequency (RR), respiratory duty cycle (Tᵢ*RR), mean inspiratory (Vt/Tᵢ) and expiratory flow (Vt/Te) and a minute ventilation (Ve);
eliminating the measurements containing abnormal values of the above-mentioned parameters, by the step of comparing each measurement with corresponding median value, calculated from the measurements available within a predetermined time window;
calculating the trends of said certain number of parameters in a predefined time period;
identifying an impending exacerbation by comparing the parameters describing said trend of said certain number of parameters with predefined thresholds;
where the step of calculating the trends of said certain number of parameters is achieved by calculation of an N order polynomial regression model; and the step of identifying an impending exacerbation by comparing said parameters describing said trends with predefined thresholds comprises the step of comparing at least one coefficient of the N order polynomial regression with predefined thresholds; wherein for each parameter of said certain number of parameters the deterioration trend of the pathology is assessed according to whether it is above or below a predefined threshold; and that it comprises the step of predicting a exacerbation by performing a weighted sum of said trend parameters (MI_{P}).
